(19) United States
(12) Patent Application Publication
(10) Pub. No.: US 2022/0008759 A1
(43) Pub. Date: Jan. 13, 2022

(54) ACTIVE RESPIRATORY OPEN FACE SHIELD SYSTEM

(71) Applicant: ZVerse, Inc., West Columbia, SC (US)

(72) Inventor: Michael J. Piatt, Springboro, OH (US)

(21) Appl. No.: 17/373,100

(22) Filed: Jul. 12, 2021

Related U.S. Application Data
(60) Provisional application No. 63/057,259, filed on Jul. 27, 2020.
(60) Provisional application No. 63/050,835, filed on Jul. 12, 2020.

(51) Int. Cl. A62B 18/00; A62B 18/08; A62B 23/02; A61F 9/02

(52) U.S. Cl. CPC A62B 18/003; A62B 18/084; A62B 23/025; A62B 18/006; A61F 9/029; A62B 23/02

(57) ABSTRACT

The present invention relates to active respiratory protective face shield systems. More specifically, the present invention is directed to face shield systems comprising a transparent lens having generally open design so to not substantially contact the user's face. The lens configured to cover at least the user's nose and mouth. An input filter system module provides a positive pressure environment to a breathing chamber by the delivery of filtered air. The positive pressure is of sufficient magnitude so to produce an air barrier for substantially preventing environmental contamination, e.g., particulate matter, microorganisms, and the like, from entering the breathing chamber. Alternate embodiments further include an output filter system module for the evacuation and substantial filtration of the user's exhalation air to surrounding environment.

### PRIORITY CLAIM AND RELATED APPLICATIONS

This non-provisional application claims the benefit of priority from the following provisional applications: U.S. Ser. No. 63/050,835 filed on Jul. 12, 2020; and provisional application U.S. Ser. No. 63/057,259 filed on Jul. 27, 2020. Said applications are incorporated by reference in their entirety.

### FIELD OF THE INVENTION

The present invention relates to active respiratory protective face shield systems. More specifically, the present invention is directed to facemask systems comprising a generally open design having a transparent lens configured to cover at least the user's nose and mouth, where at least one air filtration module is configured to filter user ambient environment air and/or the user's exhalation air.

### BACKGROUND OF THE INVENTION

Over the years the respiratory face mask technology has evolved, and standards have zo been established to quantify their performance and assure product consistency. For example, passive respiratory face masks designed to meet or exceed the OSHA N95 standard. Such face masks are commonly used by medical professionals, industrial workers, and the like. These passive masks, when properly fitted and worn, are designed to filter both the user's inhalation and exhalation air. These types of face mask designs typically do not cover the user's eyes, a potential entry point for pathogens.

Typically, N95 capable face masks are constructed from nonwoven filtration media designed to filter at least 95% of the airborne particulates entering and exiting the face mask filter, given a particle size of at least 0.3 microns. For such facemasks to be effective, it is essential that a strong seal be made with the user's nose and mouth. Over time, this intense seal and the associated tight-fitting, thin attachment straps, often lead to considerable user discomfort. The areas or causes of discomfort include: heat related issues, skin irritations, labored breathing, onset of claustrophobic feelings, and the like. Eventually, the wearer of such passive face masks will predictably experience fatigue, since they must forcefully inhale against the pressure drop of the air passing through the filter media, likewise when exhaling, must force the air out through the same filter media. Filtration materials, operating at the N95 standard, make face masks difficult to breathe through, having a pressure drop of over 0.5 inches of water across a typical filtration media. Due to the restricted air flow, the breathing air in the mask's internal volume will quickly overheat and harbor excess moisture leading to additional user discomfort. In certain circumstances the aforementioned points of user discomfort are significant factors that determine whether a worker will wear suitable protective gear or not. Moreover, due to the facial feature variations, an adequate seal between the face mask and the user may not always be possible, especially in situations where the user possesses facial hair.

Another type of passive face mask system that overcomes many of aforementioned N95 capable face mask drawbacks, is the simple transparent face shield. These shields will typically cover the user's nose, mouth, and eyes. The protective lens and support apparatus can be substantially free from contact with the user's face. Unfortunately, because these face shields possess open, unprotected perimeters, pathogens and/or particulate matter have several unobstructed paths to reach the user. These shields find utility in environments where the user is seeking protection from occupational airborne particulates or like contaminates. For example, shrapnel from industrial processes, e.g., grinding wheels; splatter and splash type contaminants from medical procedures; and the like.

It's apparent, that in the protective face mask arts, there's a long felt need for improved face mask designs or technologies that will enable a user to enjoy the advantages of an open face mask system, while also providing substantial protection from pathogens and like particulate matter.

### SUMMARY OF THE INVENTION

The present disclosure delineates an active respiratory open face shield system for providing a hygienic barrier between a user and the user's ambient environment. Other exemplary embodiments of the present invention provide others, in the user's proximate environment, substantial protection from the user. Presented are disclosures of face shields and methods of use that overcome the aforementioned disadvantages of well-known personal filtration devices and systems.

Accordingly, it is an object of the present invention to provide at least one active respiratory filtration system configured such that the system components are supported by a head harness and/or lens system. The system comprises at least one portable power source to enable mobility.

It is another object of present invention to provide at least one input filter system module attached to the rear portion of the head harness and/or the lens system. The input filter system module comprises at least one air pump, at least one input filter, at least one control system and an air transport enclosure system. The input filter system module is configured to be compact and lightweight to promote transportability.

It is yet another object of certain embodiments of present invention to provide a lens system functionally attached to the front portion of the head harness, which is configured to cooperate with one or more input filter system modules and/or output filter system modules. In certain embodiments, when both input filter system modules and output filter system modules are used, the face shield system will provide pathogenic filtering, particulate contaminant filtering, and the like, to both the user and bystanders present in the user's proximate environment. In other embodiments, where the user's environment does not contain other individuals, the use of an output filtration module is optional, and the open perimeter lens feature enables low resistance, unaltered venting of user exhalation air to surrounding environment.

It is a further object of the present invention to provide a positive pressure into the internal volume or breathing chamber of the face shield in producing an air barrier for substantially preventing environmental contamination, e.g., particulate matter, microorganisms, and the like, from entering the breathing chamber. In preferred embodiments, the volume of filtered air delivered is substantially greater than the volume of filtered air consumed (inhalation volume) by the user.

It is another object of certain embodiments of present invention to provide a face shield system where the face shield's breathing chamber includes at least one air input member configured to direct the delivery and adjust the flow patterns of filtered air presented to the user's nose and mouth.

It is yet another object of the present invention to provide air transport enclosures which are generally rectangular, configured to minimize air flow resistance, and enabling the design of compact systems.

It is yet another object of certain embodiments of present invention to provide a face shield system comprising an attachment assembly configured to engage at least a portion of the user's upper torso.

It is a further object of the present invention to provide a face shield system with at least one output filtration system module disposed onto the lens, such that exhalation air input port substantially aligns with the user's exhalation air trajectory so to diametrically engage with the user's exhalation air, thereby streamlining the evacuation and filtration of the user's exhalation air into the user's ambient environment.

It is yet another object of certain embodiments of present invention to provide input filters and/or output filters fabricated from a variety of filtration materials, including: pathogenic filtering materials, particulate contaminant filtering materials, High Efficiency Particulate Air (HEPA) certified materials, N95 capable materials, and any combination thereof. Composite filters comprising two or more layers of various filtering materials can be configured to yield enhanced air filtration results.

It is another object of this invention to provide a relatively simple system that is economical from the viewpoint of the manufacturer and consumer, is susceptible to low manufacturing costs regarding labor and materials, and which accordingly evokes low prices for the consuming public, thereby making it economically available to the buying public.

Whereas there may be many embodiments of the present invention, each embodiment may meet one or more of the foregoing recited objects in any combination. It is not intended that each embodiment will necessarily meet each objective.

Thus, having broadly outlined the more important features of the present invention in order that the detailed description thereof may be better understood, and that the present contribution to the art may be better appreciated, there are, of course, additional features of the present invention that will be described herein and will form a part of the subject matter of this specification.

In this respect, before explaining at least one embodiment of the invention in detail, it is to be understood that the invention is not limited in its application to the details of construction and the arrangements of the components set forth in the following description or illustrated in the drawings. The present invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent construction insofar as they do not depart from the spirit and scope of the conception regarded as the present invention.

### Particular Advantages of the Invention

The present invention provides cost-effective, efficient solutions directed to overcoming the disadvantages associated with uncomfortable, tight-fitting passive face masks; as well as simple, generally open, face-shield devices, which provide negligible pathogenic or particulate protection. The invention provides an open, face-shield system, free from facial contact in addition to providing filtered air to the user via an input filter system module. The filtered air provided by the input filter system module creates a positive pressure of sufficient magnitude so to produce an air barrier for substantially preventing environmental contamination, e.g., particulate matter, microorganisms, and the like, from entering the breathing chamber.

Alternate embodiments further include an output filter system module for the evacuation and filtration of the user's exhalation air to surrounding environment. One clear advantage over present-day passive masks is the elimination of the additional effort required to inhale and exhale thorough the filtration material used in such passive face mask products. The present invention provides an active means for delivering filtered air to the user; as well as the removal of exhalation air, thereby lessening the user's breathing effort.

### BRIEF DESCRIPTION OF THE DRAWINGS

The ensuing detailed description section makes reference to the annexed drawings. An enhanced understanding of the present invention will become evident when consideration is given to the detailed description thereof and objects other than the aforementioned become apparent. The invention will be described by reference to the specification and the annexed drawings, in which like numerals refer to like elements, and wherein:

FIG. 1 illustrates a general system view of an active respiratory open face shield filtration system (AROFSS) including an input filter system module (IFSM), which provides filtered air to the user.

FIG. 2 illustrates a graphical view of the breathing chamber pressure during a breathing cycle, given the positive pressure environment produced by the input filter system module (IFSM).

FIG. 3 depicts a perspective view of an exemplary input filter system module (IFSM).

FIG. 4 illustrates a perspective view of an exemplary face shield system having an input filter system module (IFSM) affixed to a user. The embodiment further depicts a streamlined air transport enclosure system comprising rectangular ducts.

FIG. 5 illustrates a front view of the face shield system shown in FIG. 4

FIG. 6 illustrates a perspective view of an exemplary face shield system including an input filter system module (IFSM) having an air transport enclosure system utilizing an air hose.

FIG. 7 illustrates a perspective view of an exemplary face shield system including an input filter system module (IFSM) integrated into the frame.

FIG. 8 illustrates a perspective view of an exemplary face shield system including an input filter system module (IFSM) integrated into the upper portion of the lens.

FIG. 9 illustrates a perspective view of an exemplary face shield system including an input filter system module (IFSM) integrated into the lower center portion of the lens.

FIG. 10 illustrates a perspective view of an exemplary face shield system including an input filter system module (IFSM) integrated into the lower, right-side portion of the lens.

FIG. 11 illustrates a general system view of an active respiratory open face shield filtration system (AROFSS) including an output filter system module (OFSM), which filters the user's exhalation air prior to release into the ambient environment.

FIG. 12 illustrates a graphical view of the breathing chamber pressure during a breathing cycle, given the positive pressure environment produced by the input filter system module (IFSM) in addition to the evacuation contribution (negative pressure) produced by the output filter system module (OFSM).

FIG. 13 illustrates a perspective view of an exemplary face shield system having an input filter system module (IFSM) affixed to a user and an output filter system module (OFSM) attached to the lens.

FIG. 14 illustrates a perspective front view of an exemplary face shield system affixed to a user. The face shield system includes an output filter system module (OFSM) attached to the lens and an air input member configured to direct the delivery and adjust the flow patterns of filtered air (produced by the IFSM) presented to the user's nose and mouth.

FIG. 15 illustrates a perspective side view of an exemplary face shield system having a headband type of attachment assembly affixed to a user. The face shield system includes an output filter system module (OFSM) attached to the lens, and input filter system module (IFSM) is attached into the headband attachment assembly.

FIG. 16 illustrates a perspective view of a respiratory face shield assembly comprising an input filter system module (IFSM) and an output filter system module (OFSM), both units affixed behind the user's head. The embodiment further depicts a streamlined air transport enclosure system comprising rectangular ducts.

### DEFINITIONS OF TERMS USED IN THIS SPECIFICATION

The active respiratory open face shield filtration system (AROFSS) discussed throughout this disclosure shall have equivalent nomenclature, including, but not limited to: the device, the system, the assembly, the face shield, the unit, the present invention, or the invention. Additionally, the term exemplary shall possess a single meaning throughout this disclosure; wherein the sole focus is directed to serving as an example, instance, or illustration. The term others or bystanders shall be defined as individuals within the immediate environment of the user, having a reasonable probability of receiving an airborne pathogen from the user. The term upper torso shall be understood to include the shoulders, neck, and any member of the head capable of providing support for the respiratory face shield systems.

The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

Note that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by”, “possessing” and “having” are all to be interpreted as open-ended terms, are all considered equivalent terms, and are used interchangeably.

### Parts/Features List

1—input filter

2—unfiltered air (ambient environment surrounding user)

3—input filter system module (IFSM), flowchart view

4—downstream (indication of filtered air flow)

6—air pump, e.g., one or more fans

8—air transport enclosure system, input (general embodiment)

10—filtered air

11—inner surface of lens 16

12—internal volume (between user 18 and inner surface 15)

13—nose and mouth of user 18

14—breathing chamber (positive pressure environment)

15—control system

16—lens (optically transparent material)

17—lens system

18—user

19—air input member (introduces filtered air 10 into breathing chamber 14)

20—unfiltered breathing chamber air or exhalation air (provides an active air barrier)

22—breathing cycle

24—exhalation pressure

26—inhalation pressure

28—minimum pressure

30—peak pressure

32—ambient pressure

34—negative pressure

36—steady state or average pressure (breathing chamber pressure when breathing is paused)

40—input filter system module (IFSM)

42—control board

44—battery (power source)

46—upper ear supports (example of an upper torso apparatus support member)

47—air transport enclosure, (rectangular embodiment)

48—respiratory face shield assembly (exemplary embodiment where only input air to the user is filtered

49—lens perimeter

50—upper torso

51—respiratory face shield system (alternate embodiment)

52—head harness

54—rear portion

56—front portion

58—lens connector (type of air input member)

60—back strap

62—air hose (type of air transport system)

64—respiratory face shield assembly (alternate embodiment)

66—frame

67—lens system

68—hinge

70—neck support member

72—output filter

74—filtered air

76—downstream

78—air pump (output)

79—output filter system module (OFSM)

80—control system

82—air transport enclosure system output (general embodiment)

84—exhalation air (unfiltered air from breathing chamber)

85—exhalation air input port (receives unfiltered air from breathing chamber)

86—excess breathing chamber air (a combination of exhalation air 84 and filtered air 19 produced by IFSM)

88—breathing chamber air (inner shield air)

90—positive pressure (produced by IFSM)

92—negative pressure (produced by OFSM)

94—steady state pressure (pressure when user breathing is paused)

96—respiratory face shield assembly (exemplary embodiment using both an IFSM and an OFSM, so that both input air to the user and user exhalation air are both filtered)

98—respiratory face shield assembly (includes both an IFSM and an OFSM, and an air input member configured to direct the delivery and adjust the flow patterns of filtered air to the user's nose and mouth

99—attachment assembly (engages neck portion of upper torso 50)

100—exhalation air trajectory

102—air input member

104—filtered air (controlled by the air input member 102)

106—respiratory face shield assembly (includes both an IFSM and an OFSM, where the input filter system module (IFSM) is attached into the headband attachment assembly)

108—headband attachment assembly

110—forehead (exemplary upper torso 50 member)

112—breathing chamber

114—breathing chamber air

116—respiratory face shield assembly (exemplary embodiment where IFSM and an OFSM systems are located behind the user's head)

### DETAILED DESCRIPTION

With reference to the drawings of the present invention, several embodiments pertaining to the faucet system of the present invention thereof will be described. In describing the embodiments illustrated in the drawings, specific terminology will be used for the sake of clarity. However, the invention is not intended to be limited to the specific terms so selected, and it is to be understood that each specific term includes all technical equivalents that operate in a similar manner to accomplish a similar purpose. Terminology of similar import other than the words specifically mentioned above likewise is to be considered as being used for purposes of convenience rather than in any limiting sense.

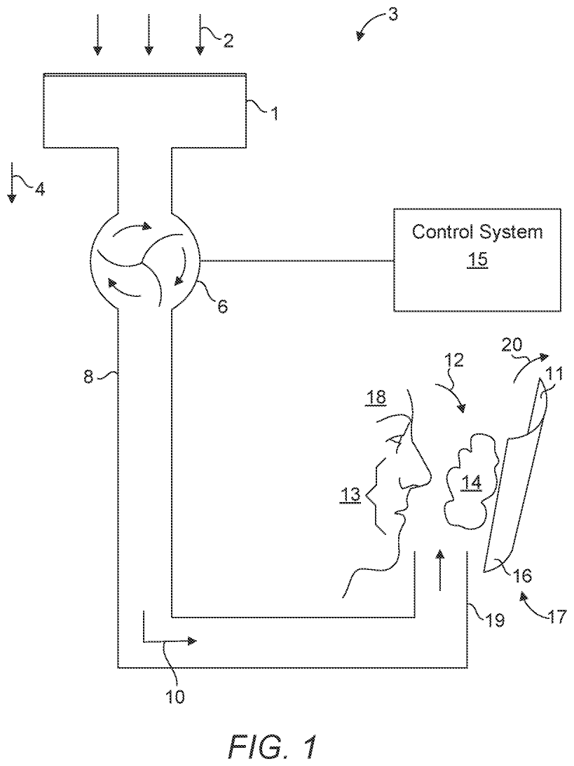

FIG. 1 illustrates a general system view of an active respiratory open face shield filtration system (AROFSS) including a flowchart view of an input filter system module (IFSM) 3, which provides filtered air 10 to user 18. Unfiltered air 2, acquired from the ambient environment surrounding user 18, is drawn through input filter 1 by air pump 6, producing filtered air 10. Air pump 6 is managed by control system 15. Filtered air 10 is directed in a downstream 4 direction through air transport enclosure system 8, exiting air input member 19 located in breathing chamber 14. Breathing chamber 14 is the approximate volume delineated by internal volume 12 formed between user's 18 face and inner surface 11 of lens 16. Control system 15 manages at least air pump 6, wherein certain embodiments are configured to regulate and monitor a variety of parameters, including: power delivery to air pump 15, velocity of air at predetermined locations throughout the system, and the like.

IFSM 3 provides filtered air 10 into breathing chamber 14 such that a positive pressure environment is created. The positive pressure environment generated within breathing chamber 14 is of sufficient magnitude so to produce a protective air barrier capable of preventing environmental contamination, e.g., particulate matter, microorganisms, and the like, from entering the breathing chamber 14. The protective air barrier is produced by unfiltered breathing chamber air 20 exiting internal volume 12 about the generally open perimeter of lens 16, flowing into the ambient surrounding environment.

The present embodiment also includes lens system 17 having a lens 16 having a least a transparent portion to enable user 18 vision. Lens 16 covers or hovers, over at least nose and mouth 13 of user's 18, and is configured so to not substantially contact user's 18 face, thereby increasing user comfort.

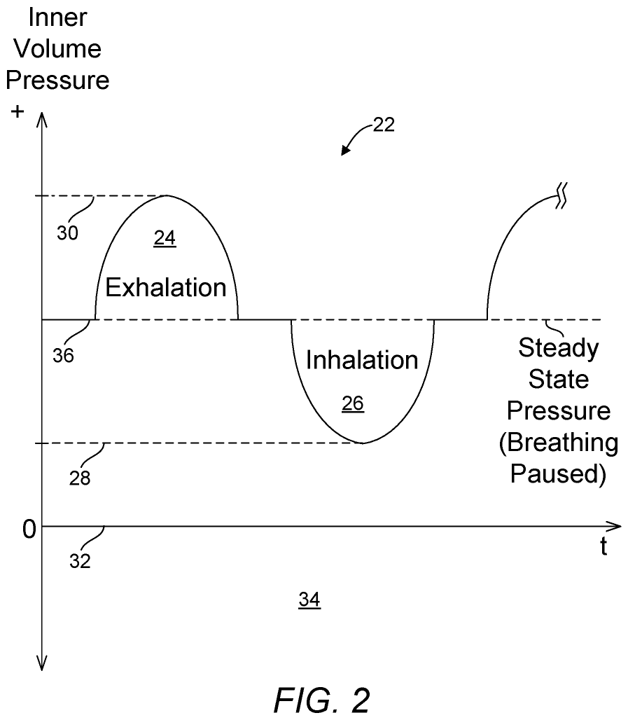

FIG. 2 illustrates a graphical view of the air pressure present within breathing chamber 14 during breathing cycle 22 of a typical user 18. The graph represents breathing chamber 14 pressure verses time. The ordinate axis depicts the ambient environmental pressure at the origin shown as ambient pressure 32; pressure values below ambient pressure 32 are represented by the area denoted as negative pressure 34. Steady state pressure 36 is produced by the delivery of filtered air 10 generated by input filter system module (IFSM) 3 superimposed over ambient pressure 32 and is understood to be the breathing chamber pressure when user 18 refrains from breathing or user's breathing is paused. Breathing cycle 22 is comprised of two primary components, the positive peak denoted as exhalation pressure 24 and the negative peak denoted as inhalation pressure 26. To ensure that unfiltered air 2 from the surrounding environment does not enter breathing chamber 14, minimum pressure 28 must be greater or equal to ambient pressure 32. As shown in FIG. 2, the value of peak pressure 30 is the summation of steady state pressure 36 and the peak pressure of exhalation pressure 24. Minimum pressure 28 point results from subtracting negative peak inhalation pressure 26 from steady state pressure 36.

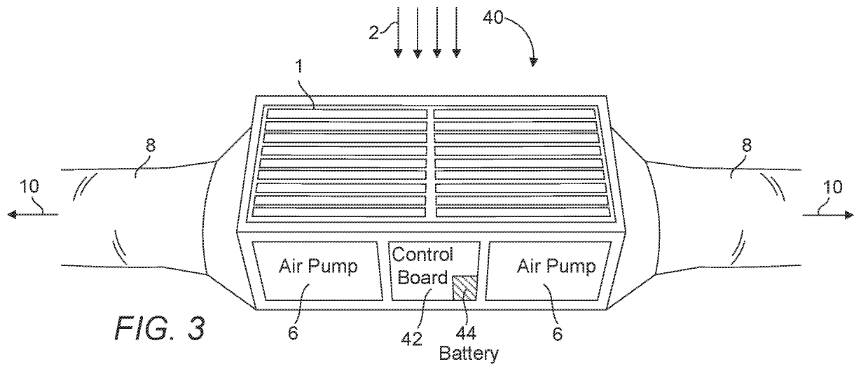

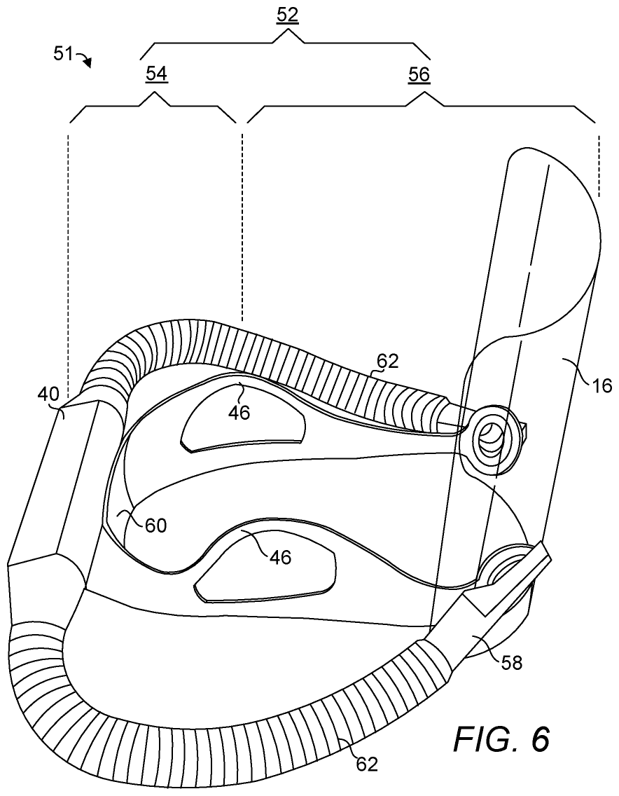

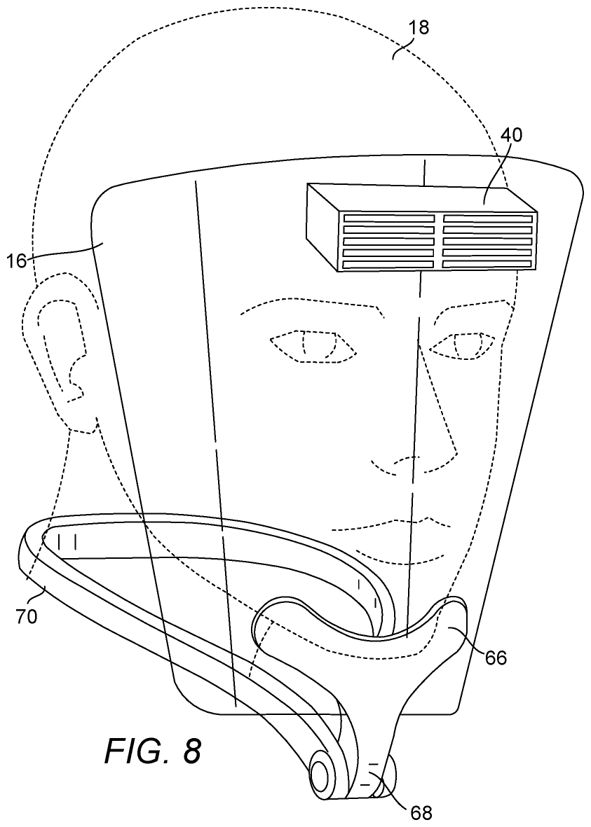

FIG. 3 illustrates a perspective view of an exemplary input filter system module (IFSM) 40. IFSM 40 provides filtered air 10 to breathing chamber 14 and can be affixed to a variety of locations, for example: to back strap 60, as shown in FIG. 6; or onto lens 16, as shown in FIGS. 8, 9, and 10. IFSM 40 is comprised of a plurality of subsystems or components which are strategically selected, and configured to be lightweight and functionally interconnect in a compact manner. IFSM 40 is comprised of at least one input filter 1, selected to perform at a predetermined level of filtration performance. Located below input filter 1 are right and left air pumps 6, which draws unfiltered air 2, taken from the user's 18 immediate environment, through input filter 1. The resulting filtered air 10 is directed to air transport enclosure system 8, or feeds directly into breathing chamber 14, or the like, depending on the specific embodiment. IFSM 40 further comprises a power source battery 44, of sufficient capacity to power the filtration system for a reasonable amount of time. Exemplary power systems include compact, high capacity, rechargeable battery systems, including supporting battery health indicators, chargers, corresponding connectors, and the like.

Exemplary IFSM 40 includes control board 42, which manages at least one air pump 6 managed by control board 42. Other exemplary control board 42 features include, but not limited to, a power management subsystem to control power delivery to air pump 6 for controlling the velocity of filtered air 10 directed into breathing chamber 14. In preferred embodiments, IFSM 40 type systems will include redundant components for safety purposes, e.g., in the event of component or system failures. Redundant components include, but are not limited to the following: air pump, rear filter, control system, and battery.

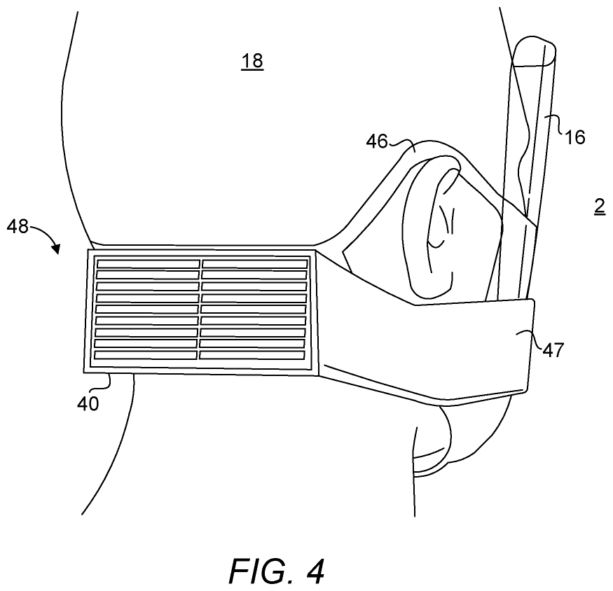

FIG. 4 illustrates a perspective view of exemplary respiratory face shield assembly 48 affixed to user 18 by a pair of upper ear supports 46. The upper ear supports 46 supportingly engage user's 18 right and left ears, the ears are just one of many support locations possible on upper torso 50. Accordingly, the term upper torso shall be understood to include the shoulders, neck, and any member or portion of the head capable of providing support for the respiratory face shield systems.

The embodiment further depicts an input filter system module (IFSM) 40 and streamlined air transport enclosure system comprising rectangular ducts 47. Rectangular ducts 47 are configured to yield a more compact and user-friendly system. In one aspect, the dimensions of rectangular ducts 47 are selected to maximize airflow, while reducing the width; the rectangular ducts 47 are elongated in the vertical direction, thereby reducing the overall width of respiratory face shield assembly 48.

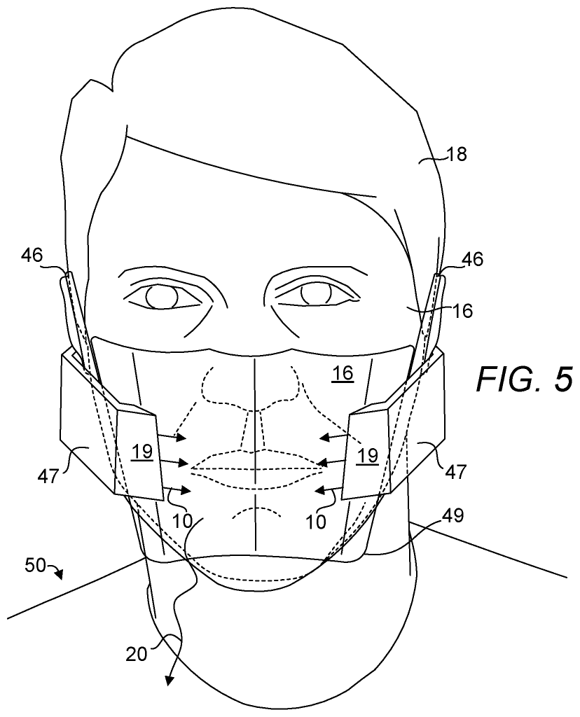

FIG. 5 illustrates a front view of the respiratory face shield assembly 48 shown in FIG. 4. This embodiment only provides filtered air 10 to user 18; exhalation air 20 from user 18 is not filtered, but freely exhausted about the open areas associated with lens perimeter 49. Exemplary right and left air input members 19 function to direct filtered air 10 into breathing chamber 14. There are several design options with respect to air input members 19. In certain embodiments, filtered air 10 can be directed through an air input member 19 having an array of smaller apertures; moreover, filtered air 10 can be steered in predetermined patterns within breathing chamber 14. Filtered air 10 predetermined patterns can be configured to accomplish one or more objectives, including: facial cooling, ease inhalation effort, minimize interference with exhalation air trajectory 100, expedite the removal of breathing chamber air 20, and the like.

FIG. 6 illustrates a perspective view of an exemplary face shield system 51 including an input filter system module (IFSM) 40 having an air transport enclosure system utilizing an air hose 62 type system. The face shield components are organized and supported on head harness 52, which is configured to securely attach to user's 18 head. Head harness 52 further comprises back strap 60, which optionally includes a means for separating the strap into two sections to expedite attaching and/or removal of face shield system 51 to user 18; as well as expediting disassembly of face shield system 51 components for service, repair, cleaning, and the like.

Head harness 52 is comprised of two contiguous key sections, front portion 56, and rear portion 54. Rear portion 54 includes back strap 60, which provides support for IFSM 40. Front portion 56 comprises lens system 17, containing lens 16 and lens connector 58. IFSM 40 is connected to lens connector 58 via air hose 62. In exemplary embodiment face shield system 51, system support is provided by a pair of right and left upper ear supports 46, configured to correspondingly engage the right and left ears of user 18.

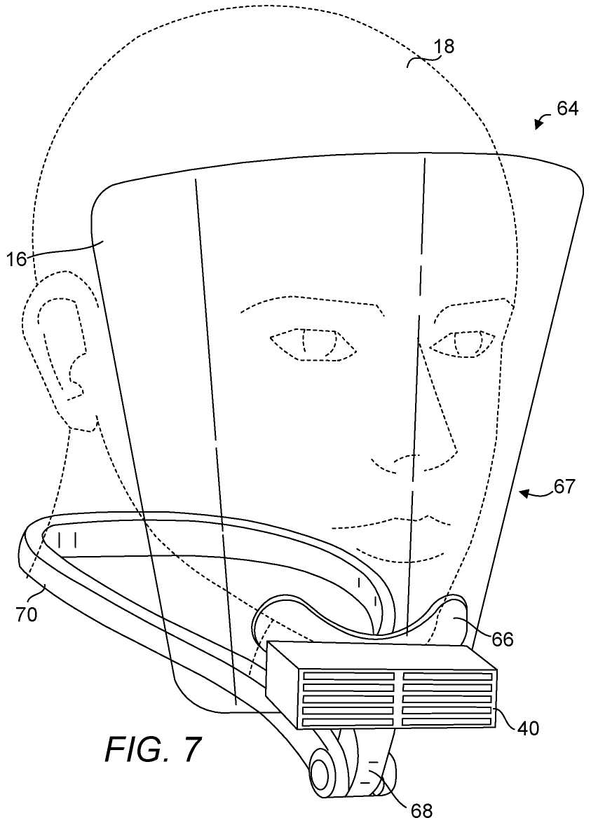

FIG. 7 illustrates a perspective view of respiratory face shield assembly 64. Face shield assembly 64 is yet another embodiment of an active respiratory open face shield filtration system including an input filter system module (IFSM) 40, which provides filtered air 10 to the user. Respiratory face shield assembly 64 includes an IFSM 40 integrated into front portion of frame 66. The front portion of frame 66, which includes hinge 68 for supporting lens system 67; lens system 67 includes lens 16, which covers user's 18 face, e.g., nose, mouth, and eyes. The rear portion of frame 66 provides upper torso 50 support, in the form of neck support member 70.

Hinge 68 enables user 18 to hingedly move lens 16 away from user's 18 face, while neck support member 70 continues to provide substantial support for shield assembly 64 system as a whole. In the present embodiment, since IFSM 40 is integrated into front portion of frame 66, filtered air 10 is immediately directed into the breathing chamber, sans an air transport enclosure system.

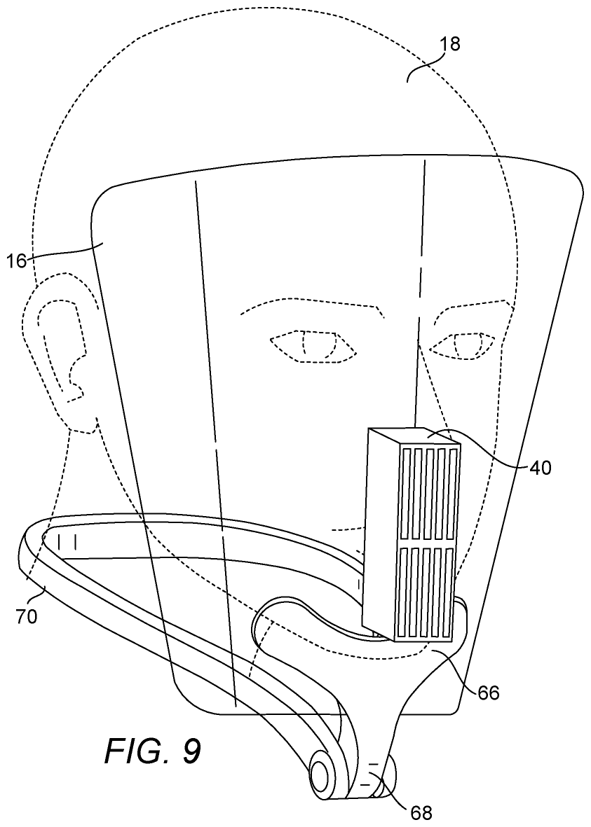

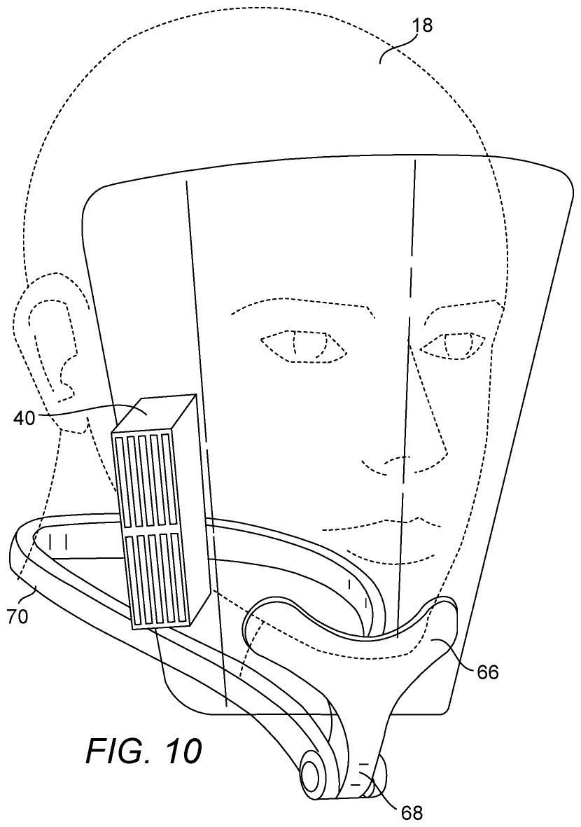

FIGS. 8, 9, and 10 illustrate various embodiments based on the respiratory face shield assembly 64 shown in FIG. 7. All the embodiments shown in FIGS. 8, 9, and 10 have an input filter system module (IFSM) 40 mounted into lens 16, but at different locations. Again, since IFSM 40 is integrated directly into lens 16, filtered air 10 is immediately directed into the breathing chamber, and therefore does not require any type of air transport enclosure system 8 to guide filtered air 10. FIG. 8 illustrates a face shield system where IFSM 40 is integrated into the upper portion of lens 16. FIG. 9 illustrates a face shield system where IFSM 40 is integrated into the lower, center portion of lens 16. FIG. 10 illustrates a face shield system where IFSM 40 is integrated into the lower, right-side portion of lens 16.

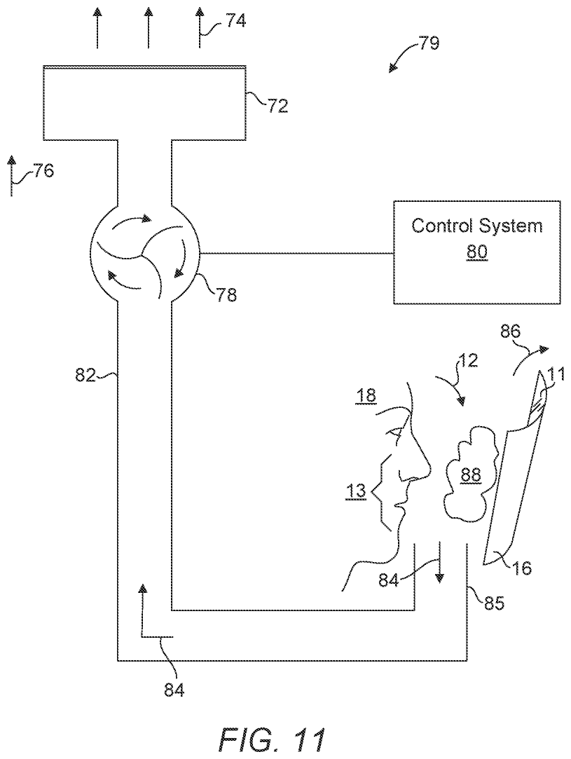

FIG. 11 illustrates a general system view of primarily the output filter system module (OFSM) 79 portion of an exemplary active respiratory open face shield filtration system (AROFSS). OFSM 79, in addition to the aforementioned input filter system module (IFSM) 3 system; provides a means for filtering user's 18 exhalation air 84 prior to release into the ambient environment, thereby protecting bystanders, materials, and the like. It is understood breathing chamber air 88 is comprised of at least exhalation air 84 and filtered air 10. Given the proper configuration/calibrations, there's little to no excess breathing chamber air 86 escaping about the open perimeter of lens 16.

OFSM 79 portion of FIG. 11 delineates the apparatus and process for filtering user's 18 exhalation air 84 prior to release into the ambient environment. OFSM 79 exhausts filtered air 74 into the ambient environment; accordingly, unfiltered breathing chamber air 88 is motivated in a downstream 76 direction by air pump 78. Starting from user 18, breathing chamber air 88 is pulled through exhalation air input port 85 and channeled through air transport enclosure system 82 where breathing chamber air 88 is forced through the receiving or input portion of output filter 72 by air pump 78, resulting in filtered air 74 exiting output filter 72 into the ambient environment. In certain embodiments, determined by the specific mounting location of OFSM 79, an air transport enclosure system 82 may not be necessary. Control system 80 manages at least air pump 78, wherein certain embodiments are configured to regulate and monitor a variety of parameters, including: power delivery to air pump 78, velocity of air throughout the system, and the like.

In preferred embodiments, the OFSM 79 will include redundant components for safety, in case of component failures. Redundant components include, but are not limited to the: air pump, output filter, control system, and battery. As delineated in the in the previous discussions associated with FIG. 1, embodiments including OFSM 79 also comprises a lens 16 for covering or hovering over at least nose and mouth 13 of user's 18, and is also configured so to not substantially contact user's 18 face, promoting increased user comfort.

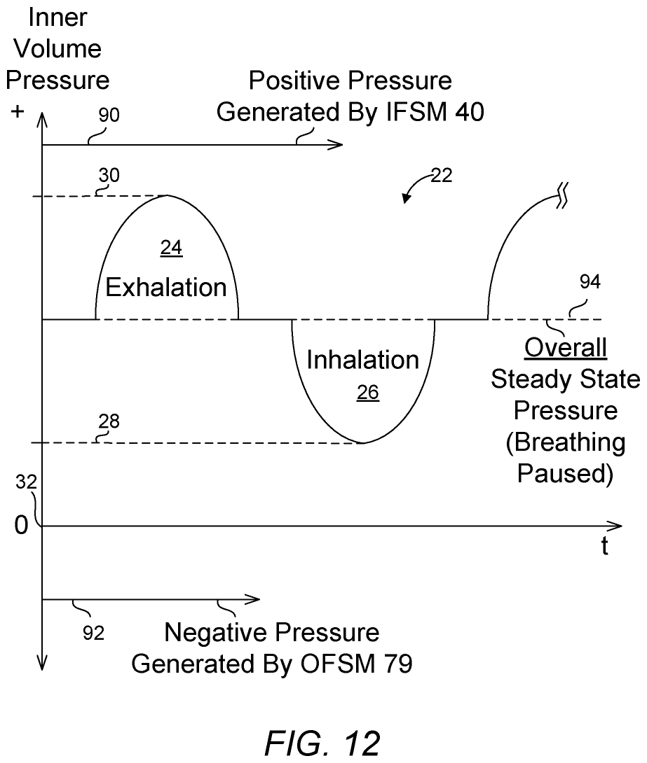

FIG. 12 illustrates a graphical view of the air pressure within breathing chamber 88 during breathing cycle 22 of a typical user 18, given an embodiment of the present invention that includes both input filter system module (IFSM) 40—for filtering inhalation air, and an output filter system module (OFSM) 79—for filtering exhalation air. The graph represents breathing chamber 88 pressure verses time. The ordinate axis, at its origin, depicts the ambient environmental pressure as ambient pressure 32. The area of negative pressure is located in the area below ambient pressure 32.

Steady state, positive pressure 90 is produced by the generation of filtered air 10 by IFSM 40. Steady state, negative pressure 92 is produced by the evacuation of exhalation air 84 processed by OFSM 79.

Steady state pressure 94 is the difference resulting from subtracting negative pressure 92 from positive pressure 90 and is understood to be the breathing chamber 88 pressure when user 18 refrains from breathing or breathing is paused (e.g., the pause between inhalation and exhalation cycles 22). Breathing cycle 22 is comprised of two primary components, the positive peak denoted as exhalation pressure 24 and the negative peak denoted as inhalation pressure 26. In order to ensure that unfiltered air from the surrounding environment does not enter breathing chamber 88, minimum pressure 28 must be greater or equal to ambient pressure 32. As depicted in FIG. 2, the value of peak pressure 30 is the summation of steady state pressure 94 and the peak value of exhalation pressure 24. Minimum pressure 28 is the difference resulting from subtracting the negative peak value of inhalation pressure 26 from steady state pressure 94. The overall pressure created in breathing chamber 88 is configured to be positive. The positive pressure environment generated is of sufficient magnitude so to produce a protective air barrier, capable of preventing environmental contamination, e.g., particulate matter, microorganisms, and the like, from entering the breathing chamber 88. The protective air barrier is produced by filtered air 10 continuously flowing into breathing chamber air 20 and exiting internal volume 12 about the generally open perimeter of lens 16.

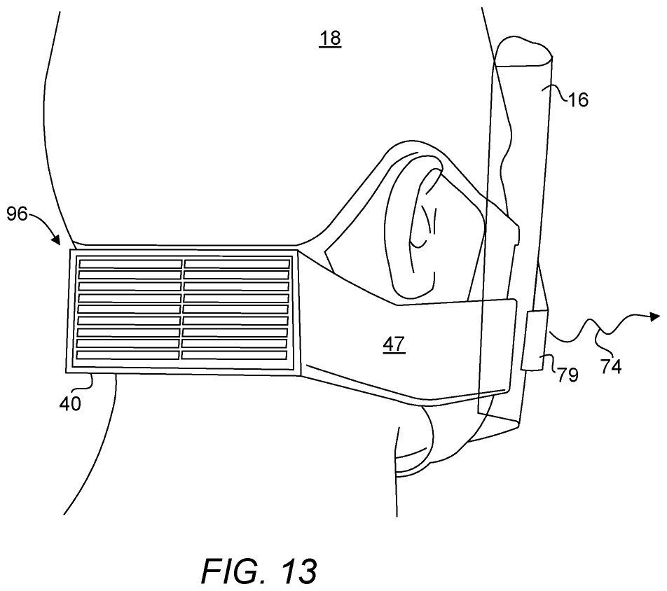

FIG. 13 illustrates a perspective rear-side view of an exemplary respiratory face shield assembly 96, which follows the teachings associated with FIGS. 11 and 12. Assembly 96, shown affixed to user 18, includes an input filter system module (IFSM) 40 engaging the back portion of user's 18 head, and an output filter system module (OFSM) 79 attached to lens 16. Again, the present embodiment includes both IFSM 40—for filtering inhalation air; in addition to OFSM 79—for filtering exhalation air 74, to protect bystanders and the like in the user's environment. The present embodiment also includes lens 16 having a least a transparent portion to enable user 18 vision. Lens 16 covers or hovers, over at least nose and mouth 13 of user's 18, and is configured so to not substantially contact user's 18 face, thereby providing enhanced user comfort.

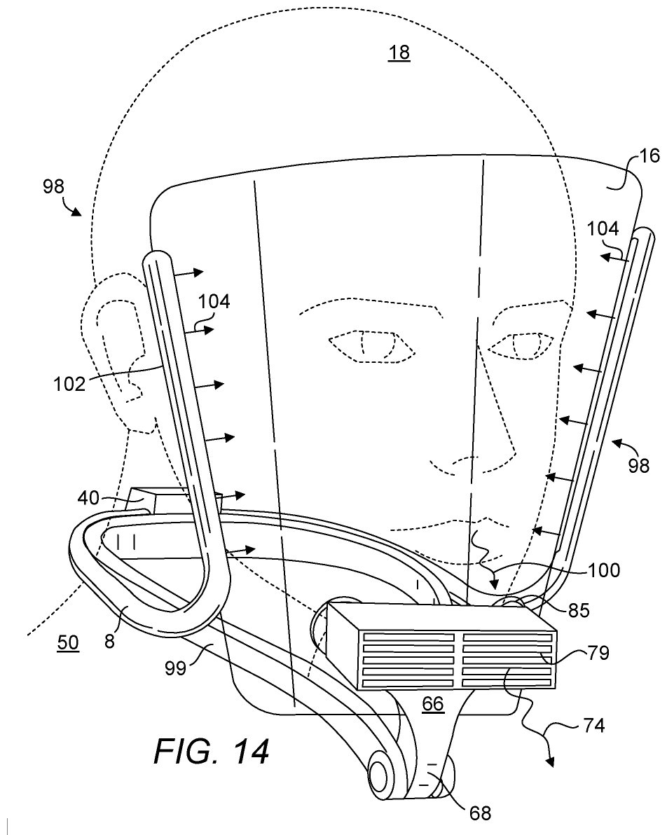

FIG. 14 illustrates a perspective front view of an exemplary respiratory face shield assembly 98 affixed to user 18 via a generally circular attachment assembly 99 configured to engage the neck portion of upper torso 50. The respiratory face shield assembly 98 includes an output filter system module (OFSM) 79 attached to frame 66. Input filter system module (IFSM) 40 is coupled to air transport enclosure system 8, further attached to air input member 102, which is configured to direct the delivery and associated air flow patterns so to present filtered air 104 to the user's nose and mouth 13 in a predetermined manner. Predetermined patterns include air flows that enhance facial cooling, expedite the removal of exhalation air, and the like.

Assembly 98 further depicts OFSM 79 functionally attached onto frame 66 to provide an exhalation air input port 85 for user 18; in other embodiments, OFSM 79 can be attached directly onto lens 16, frame 66, or like surfaces. In this and other preferred embodiments, exhalation air input port 85 substantially aligns with the user 18 exhalation air trajectory 100 so to efficiently engage with the user's exhalation air, thereby streamlining the evacuation and filtration of the user's 18 exhalation air into the user's ambient environment. In preferred embodiments, there is a diametric or linear (shortest path option) exhaust path from user's nose and mouth 13 to exhalation air input port 85.

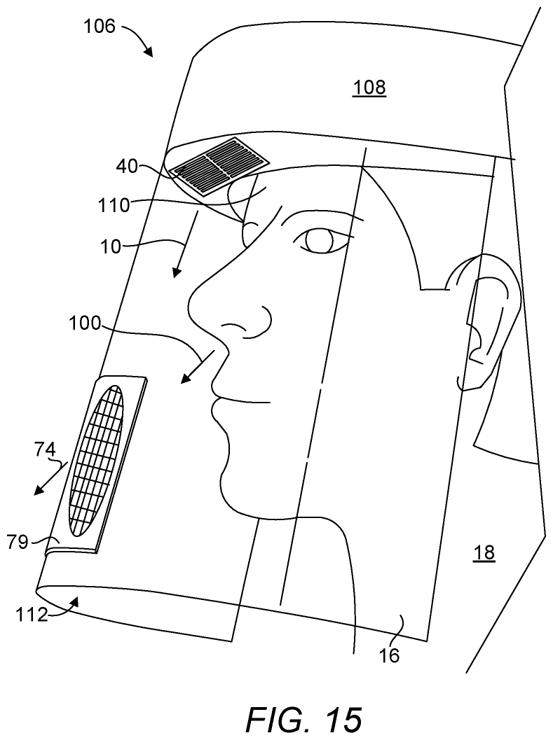

FIG. 15 illustrates a perspective side view of respiratory face shield assembly 106, having headband attachment assembly 108 configured to engage forehead 110 portion of user's 18 head. The exhalation air input port 85 portion of output filter system module (OFSM) 79 is directly attached onto lens 16, such that the exhalation air input port 85 substantially aligns with the user 18 exhalation air trajectory 100, so to efficiently engage with the user's exhalation air for streamlining the evacuation and filtration of user's 18 exhalation air into the user's ambient environment. In preferred embodiments there's a diametric or linear (shortest path option) exhaust path from user's nose and mouth 13 area to exhalation air input port located on OFSM 79. Input filter system module (IFSM) 40 is fastened onto the headband attachment assembly 108. IFSM 40 delivers filtered air 10 into breathing chamber 112 from above nose and mouth 13 areas, thereby providing cooling airflow to a substantial portion of user's 18 face.

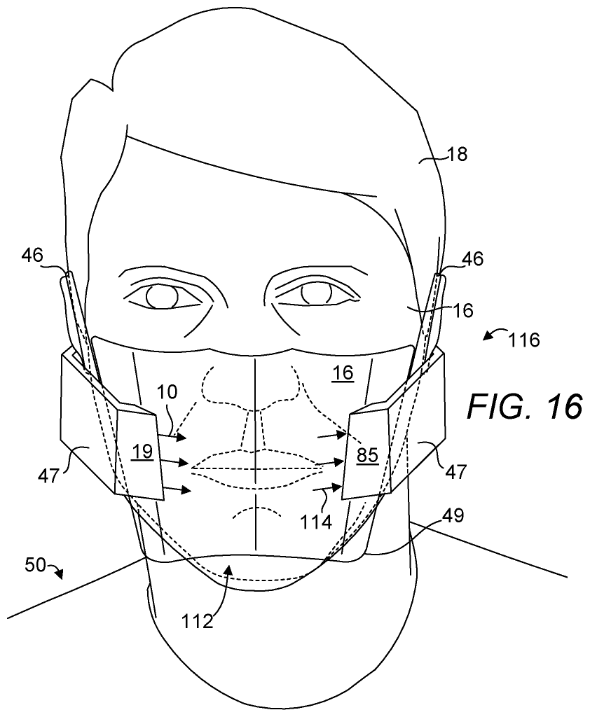

FIG. 16 illustrates a perspective view of respiratory face shield assembly 116 comprising both an input filter system module (IFSM) 40 and an output filter system module (OFSM) 79, where both units affixed behind the user's head. The functions of IFSM 40 and OFSM 79 are best delineated in FIGS. 1 and 11, respectively. It is understood that both IFSM 40 and OFSM 79 do not have to exist as separate systems; The components and corresponding functions provided by the two separate systems can be combined into a more streamlined system. Moreover, in some embodiments, the IFSM 40 and OFSM 79 systems/functions or can share one or more components.

Depicted is a streamlined air transport enclosure 47 type system, utilizing rectangular ducts. Air input member 19 located on the right side of user 18 is attached to an IFSM 40 via air transport enclosure 47 and serves to direct filtered air 10 into the right portion of breathing chamber 112. Exhalation air input port 85 located on the left side of user 18 is attached to OFSM 79 via opposing air transport enclosure 47 and serves to extract breathing chamber air 114 from breathing chamber 112, filter the extracted air, and dissipate the filtered air into the surrounding environment.

Face shield assembly 116 is configured having two opposing members for the creation of a predetermined air curtain drawn across the nose and mouth 13 of user 18. The two opposing members include air input member 19, and exhalation air input port 85. Air input member 19—powered by IFSM 40, and exhalation air input port 85—powered by OFSM 79 are systems configured to cooperate with each other so to create a self-contained breathing chamber 112, as depicted in FIG. 12. In preferred embodiments, self-contained breathing chamber 112, IFSM 40, OFSM 79, and supporting components are configured such that the predetermined air curtain produced, engages with user 18 such that the ambient unfiltered air surrounding user 18 will not be drawn into breathing chamber 112, and neither will user's 18 exhalation air be exhausted directly into the ambient environment without proper filtration.

1. A face shield system for providing a hygienic barrier between a user and the user's ambient environment, said face shield system comprising a harness adapted to engage with a user's head, said harness comprising:
(a) a rear portion comprising an input filtration system module attached thereon, said input filtration system module further comprising:
(i) an air pump for moving ambient unfiltered air through an input filter so to produce a delivery of filtered air;
(ii) a control system functionally connected to said air pump for controlling said delivery of filtered air, said control system further comprising a power source for motivating said air pump; and
(iii) an air transport enclosure system configured for directing flow of said delivery of filtered air exiting said input filtration system module; and
(b) a front portion comprising a lens system supported thereon configured to cover at least the user's nose and mouth, said lens system comprising:
(i) a lens, without any means for facial sealing, said lens having an inner and outer surface, wherein at least a portion of said lens is configured from a transparent material; said lens is adapted to hover over at least the user's nose and mouth so to substantially form an internal volume between said lens's inner surface and the user, delineating the disposition of a breathing chamber; and
(ii) at least one air input member for said delivery of filtered air into said breathing chamber, wherein said at least one air input member is fluidly attached to said air transport enclosure system.

2. The face shield system of claim 1, wherein said breathing chamber receives said delivery of filtered air of sufficient magnitude so to produce a positive pressure environment within said breathing chamber, thereby producing an active air barrier configured to substantially block ambient environmental contaminants from entering said breathing chamber.

3. The face shield system of claim 1, wherein said input filter is fabricated from a material selected from the group consisting of a pathogenic filtering material, a particulate contaminant filtering material, a High Efficiency Particulate Air (HEPA) certified material and any combination thereof.

4. (canceled)

5. The face shield system of claim 1, wherein said air transport enclosure system is selected from the group consisting of a hose, a tube, a rectangular duct and any combination thereof.

6. The face shield system of claim 1, further comprising at least one output filtration system module for the evacuation and filtration of the user's exhalation air from said breathing chamber, said output filtration system module is functionally disposed onto said lens, said output filtration system module comprising:
(a) an air pump for moving unfiltered exhalation air from said breathing chamber through an output filter, so to produce a delivery of filtered exhalation air into the ambient environment;
(b) a control system functionally connected to said air pump for controlling said delivery of filtered exhalation air, said control system further comprising a power source for motivating said air pump; and
(c) an exhalation air input port configured for receiving the user's exhalation air from said breathing chamber.

7. The face shield system of claim 6, further comprising said at least one output filtration system module that is functionally attached onto a portion of said lens such that said exhalation air input port substantially aligns with the user's exhalation air trajectory so to diametrically engage with the user's exhalation air, thereby streamlining the evacuation and filtration of the user's exhalation air into the user's ambient environment.

8. A face shield system for providing a hygienic barrier between a user and the user's ambient environment, said face shield system comprising an attachment assembly configured to engage at least a portion of the user's upper torso for providing support for said face shield system; and a lens system comprising a lens without any means for facial sealing, and a mechanism for supportingly attaching said lens to said attachment assembly, said lens comprising:
(a) an inner and outer surface, further comprising at least a portion of said lens is configured from a substantially transparent material; said lens is adapted to hover over at least the user's nose and mouth so to substantially form an internal volume between said lens's inner surface and the user, delineating the disposition of a breathing chamber; and
(b) at least one input filtration system module attached thereon, said input filtration system module further comprising:
(i) an air pump for moving ambient unfiltered air through an input filter, so to produce a delivery of filtered air;
(ii) a control system functionally connected to said air pump for controlling said delivery of filtered air, said control system further comprising a power source for motivating said air pump; and
(iii) a filtered air output port configured for directing flow of said delivery of filtered air into said breathing chamber.

9. The face shield system of claim 8, further comprising at least one output filtration system module for the evacuation and filtration of the user's exhalation air from said breathing chamber to the ambient environment, said output filtration system module is functionally disposed onto said lens, said output filtration system module comprising:
(a) an air pump for moving unfiltered exhalation air from said breathing chamber through an output filter so to produce a delivery of filtered exhalation air into the ambient environment;
(b) a control system functionally connected to said air pump for controlling said delivery of filtered exhalation air, said control system further comprising a power source for motivating said air pump; and
(c) an exhalation air input port configured for receiving the user's exhalation air disposed into said breathing chamber.

10. The face shield system of claim 9, further comprising said at least one output filtration system module that is functionally attached onto a portion of said lens such that said exhalation air input port substantially aligns with the user's exhalation air trajectory so to diametrically engage with the use's exhalation air, thereby streamlining the evacuation and filtration of the user's exhalation air into the user's ambient environment.

11. The face shield system of claim 9, wherein said output filter is fabricated from a material selected from the group consisting of a pathogenic filtering material, a particulate contaminant filtering material, a High Efficiency Particulate Air (HEPA) certified material and any combination thereof.

12. The face shield system of claim 8, wherein said breathing chamber receives said delivery of filtered air of sufficient magnitude so to produce a positive pressure environment within said breathing chamber, thereby producing an active air barrier configured to substantially block ambient environmental contaminants from entering said breathing chamber.

13. The face shield system of claim 8, wherein said input filter is fabricated from a material selected from the group consisting of a pathogenic filtering material, a particulate contaminant filtering material, a HEPA certified material and any combination thereof.

14. The face shield system of claim 8, wherein said attachment assembly comprises at least one hinge, enabling the user to hingedly move said lens away from at least the user's nose and mouth.
